(19) United States
(12) Patent Application Publication
(10) Pub. No.: US 2026/0124159 A1
(43) Pub. Date: May 7, 2026

(54) FAT-SOLUBLE VITAMIN POWDERS

(71) Applicant: DSM IP ASSETS B.V., Maastricht (NL)

(72) Inventors: Gabriela BADOLATO BÖNISCH, Kaiseraugst (CH); Casper DE BOER, Kaiseraugst (CH); Thomas LINDEMANN, Kaiseraugst (CH); Fabiola PORTA, Kaiseraugst (CH); Christian SCHAEFER, Kaiseraugst (CH); Bernd SCHLEGEL, Kaiseraugst (CH); Ralph SCHNEITER, Kaiseraugst (CH); Christos TSEKOU, Kaiseraugst (CH); Olivia Brigitte VIDONI, Kaiseraugst (CH); Dirk Oliver WOERNE, Kaiseraugst (CH)

(21) Appl. No.: 19/105,211

(22) PCT Filed: Aug. 18, 2023
(86) PCT No.: PCT/EP2023/072840

§ 371 Date: Feb. 20, 2025

(30) Foreign Application Priority Data
Aug. 24, 2022 (EP) 22191964.0

(51) Int. Cl. A61K 31/07; A61K 9/16

(52) U.S. Cl. CPC A61K 31/07; A61K 9/1617; A61K 9/1652

(57) ABSTRACT

The present invention relates to a formulation, which comprises a fat-soluble vitamin and which can be used for fortifying food products (especially staple food).

The present invention relates to a formulation, which comprises a fat-soluble vitamin and which can be used for fortifying food products (preferably staple food, especially flour).

Examples of fat-soluble vitamins are vitamin A, vitamin D, Vitamin K and/or its derivatives or mixtures thereof. Preferred is vitamin A and/or its derivative, more preferred vitamin A esters and here especially the vitamin A palmitate.

Vitamin A is an essential micronutrient whose deficiency is still a major health concern in many regions of the world. It plays an essential role in human growth and development, immunity, and vision, but may also help prevent several other chronic diseases. The total amount of vitamin A in the human diet often falls below the recommended dietary allowance of approximately 900-1000 μg/day for a healthy adult. Moreover, a significant proportion of vitamin A may be degraded during food processing, storage, and distribution, thereby reducing its bioactivity. Finally, the vitamin A in some foods has a relatively low bioavailability, which further reduces its efficacy. The World Health Organization has recommended fortification of foods and beverages as a safe and cost-effective means of addressing vitamin A deficiency. However, there are several factors that must be overcome before effective fortified foods can be developed, including the low solubility, chemical stability, and bioavailability of this oil-soluble vitamin. Consequently, strategies are required to evenly disperse the vitamin throughout food matrices, to inhibit its chemical degradation, to avoid any adverse interactions with any other food components, to ensure the food is palatable, and to increase its bioavailability.

Therefore, there is always a need for formulation, which can be used for food fortification.

The problem of fat-soluble vitamins is that they are prone to oxidize. This means that the content of these vitamins decreases during storage (as well as during the production of the food product). This is a problem in the production of formulation comprising these vitamins as well as during the storage of product comprising these vitamins.

To obtain and keep a certain amount of these ingredients in the final product, a usual way is to add these vitamins in excess.

Another way to solve that problem, antioxidants can be added to the vitamins. A very common and usual antioxidant is BHT (butyl hydroxy toluene). At the moment a majority of vitamin A powders on the market intended for flour or sugar fortification contain BHT.

BHT (butylated hydroxytoluene) is a lab-made chemical that is added to foods as a preservative, but it has a negative reputation both from a health and an environmental perspective.

For that reason there is a desire to avoid BHT in food products (or in formulations, which are used in food products).

Therefore, the goal of the present invention was to find a formulation approach with a high amount of at least one fat-soluble vitamin, which does not comprise BHT and which is free of any disaccharide. Such a formulation needs to be stable, easy to be produced and which can be used in a large variety of (food) products.

Surprisingly, it was found that a solid formulation which comprises
(i) at least one fat soluble vitamin, and
(ii) at least one hydrocolloid, and
(iii) optionally at least one oil, and
(iv) tocopherol and
(v) sodium ascorbate,
with the proviso that the formulation does not comprise
(a) any BHT, and
(b) any starch hydrolysate, and
(c) any disaccharide,
shows all the advantages as listed above.

Therefore, the present invention relates to a solid formulation (SF) comprising
(i) at least one fat soluble vitamin, and
(ii) at least one hydrocolloid, and
(iii) optionally at least one oil, and
(iv) tocopherol and
(v) sodium ascorbate,
with the proviso that the formulation does not comprise
(a) any BHT, and
(b) any starch hydrolysate, and
(c) any disaccharide.

Preferably, the solid formulation according to the present invention does not comprise any sucrose.

Therefore, the present invention relates to a solid formulation (SF′), which is the solid formulation (SF), which is free of any disaccharide chosen from the group consisting of sucrose, lactose, trehalose, cellobiose and chitobiose (preferably sucrose).

Therefore, the present invention relates to a solid formulation (SF″), which is the solid formulation (SF), which is free of sucrose.

The solid formulation according to the present invention does not comprise any BHT or any starch hydrolysate or optionally any disaccharide. This means that the solid is (substantially) free from BHT as well as from any starch hydrolysate and preferably also free from any disaccharide.

It might be that traces of these compounds may be present in the formulation. But they are not added intentionally. It may be that traces of such compounds are present in the ingredients (i) to (v) or in other ingredients, which may be part of the solid formulation according to the present invention.

It was surprisingly shown that the formulations according to the present invention have similar or even improved stability compared to other formulations containing various amount of starch hydrolysate.

Due to fact that usually the formulation for that purpose have the starch hydrolysate and/or BHT in it, the present formula is less complexity land less hygroscopic and also easier to be used because of less ingredients.

As stated above the formulation according to the present invention are free of BHT and free of any starch hydrolysate.

Hydrolyzed starches are the dried products or aqueous dispersions of saccharides (hydrolysates) obtained by hydrolysis of native starch by using suitable acid or enzymes. Starch hydrolysates denote dextrins, maltodextrins and glucose syrup

Therefore, the present invention relates to a solid formulation (SF1), which is the solid formulation (SF), (SF′) or (SF″), which is free of any dextrin, any maltodextrin and any glucose syrup.

The formulation according to the present invention comprises at least one fat-soluble vitamin. Fat-soluble vitamins are vitamin A, vitamin D, Vitamin K and/or its derivatives or mixtures thereof. Preferred is vitamin A and/or its derivative, more preferred are vitamin A esters and especially preferred is vitamin A palmitate.

Therefore, the present invention relates to a solid formulation (SF2), which is the solid formulation (SF), (SF′), (SF″) or (SF1), wherein the at least one fat-soluble vitamin is chosen from the group consisting of vitamin A, vitamin D, Vitamin K and its derivatives thereof.

Therefore, the present invention relates to a solid formulation (SF2′), which is the solid formulation (SF), (SF′), (SF″) or (SF1), wherein at least one fat-soluble vitamin is chosen from the group consisting of vitamin A and derivatives thereof.

Therefore, the present invention relates to a solid formulation (SF2″), which is the solid formulation (SF), (SF′), (SF″) or (SF1), wherein the fat-soluble vitamin is a vitamin A ester.

Therefore, the present invention relates to a solid formulation (SF2″), which is the solid formulation (SF), (SF′), (SF″) or (SF1), wherein the fat-soluble vitamin is a vitamin A palmitate.

The content of the at least one fat-soluble vitamin in the solid formulation is usually from 3 to 30 weight-% (wt.-%), based on the total weight of the solid formulation. Preferably from 5 to 25 wt.-%, more preferably from 10 to 25 wt.-%, based on the total weight of the solid formulation.

Therefore, the present invention relates to a solid formulation (SF3), which is the solid formulation (SF), (SF′), (SF″), (SF1), (SF2), (SF2′), (SF2″) or (SF2″), wherein the content of the at least one fat-soluble vitamin is from 3 to 30 weight-% (wt.-%), based on the total weight of the solid formulation.

Therefore, the present invention relates to a solid formulation (SF3′), which is the solid formulation (SF), (SF′), (SF″), (SF1), (SF2), (SF2′), (SF2″) or (SF2″), wherein the content of the at least one fat-soluble vitamin is from 5 to 25 wt.-%.

Therefore, the present invention relates to a solid formulation (SF3″), which is the solid formulation (SF), (SF′), (SF″), (SF1), (SF2), (SF2′), (SF2″) or (SF2″), wherein the content of the at least one fat-soluble vitamin is from 10 to 25 wt.-%, based on the total weight of the solid formulation.

The solid formulation according to the present invention comprises at least one hydrocolloid.

In the context of the present invention, hydrocolloids are polysaccharides, gelatin of low bloom, medium bloom or high bloom from fish, pork or bovine, caseines/caseinates and other proteinaceous hydrocolloids.

Preferred hydrocolloids according to the invention are polysaccharides.

In the context of the present invention, the term polysaccharide as used herein includes xanthan gum, gum acacia, pectin, guar gum, caroub gum, alginates, celluloses, cellulose derivatives, starch and starch derivatives. Preferred polysaccharides according to the present invention are gum acacia, starch, starch derivatives, more preferred are gelatinized starch and modified food starch and especially preferred are modified food starches.

In the context of the present invention, the term “modified food starch” as used herein relates to modified starches that are made from starches substituted by known chemical methods with hydrophobic moieties. For example starch may be treated with cyclic dicarboxylic acid anhydrides such as succinic and/or glutaric anhydrides, substituted with an alkyl or alkenyl hydrocarbon group.

A very common and preferred modified starch is starch sodium octenyl succinate (OSA-starch). OSA-starch as used herein denotes any starch (from any natural source such as corn, wheat, tapioca, potato or synthesized) that was treated with octenyl succinic anhydride. The degree of substitution, i.e. the number of esterified hydroxyl groups with regard to the total number of hydroxyl groups usually varies in a range of from 0.1 percent to 10 percent, preferably in a range of from 0.5 percent to 5 percent, more preferably in a range of from 2 percent to 4.5 percent.

OSA-starches are commercially available e.g. from Ingredion under the trade names HiCap 100, Capsul HF, Capsul HS, Purity Gum 2000, UNI-PURE, HYLON VII from Roquette Freres; from Cargill under the trade name C*EmCap or from Tate and Lyle.

The content of the at least one hydrocolloid in the solid formulation is usually from 25 to 85 wt.-%, based on the total weight of the solid formulation, preferably from 25 to 80 wt.-%, more preferably from 25 to 75 wt.-%, based on the total weight of the solid formulation.

Therefore, the present invention relates to a solid formulation (SF4), which is the solid formulation (SF), (SF′), (SF″), (SF1), (SF2), (SF2′), (SF2″), (SF2″), (SF3), (SF3′) or (SF3″), wherein the at least one hydrocolloid is chosen from the group consisting of polysaccharides, gelatin of low bloom, medium bloom or high bloom from fish, pork or bovine, caseines/caseinates and other proteinaceous hydrocolloids.

Therefore, the present invention relates to a solid formulation (SF4′), which is the solid formulation (SF), (SF′), (SF″), (SF1), (SF2), (SF2′), (SF2″), (SF2′″), (SF3), (SF3′) or (SF3″), wherein the at least one hydrocolloid is chosen from the group consisting of xanthan gum, gum acacia, pectin, guar gum, caroub gum, alginates, celluloses, cellulose derivatives, starch and starch derivatives.

Therefore, the present invention relates to a solid formulation (SF4″), which is the solid formulation (SF), (SF′), (SF″), (SF1), (SF2), (SF2′), (SF2″), (SF2′″), (SF3), (SF3′) or (SF3″), wherein the hydrocolloid is chosen from the group consisting of modified food starch, xanthan gum, gum acacia, pectin, guar gum and caroub gum.

Therefore, the present invention relates to a solid formulation (SF4″), which is the solid formulation (SF4″), wherein the modified food starch is starch sodium octenyl succinate.

Therefore, the present invention relates to a solid formulation (SF5), which is the solid formulation (SF), (SF′), (SF″), (SF1), (SF2), (SF2′), (SF2″), (SF2″), (SF3), (SF3′), (SF3″), (SF4), (SF4′), (SF4″) or (SF4″), wherein the content of the at least one hydrocolloid is 25 to 85 wt.-%, based on the total weight of the solid formulation.

Therefore, the present invention relates to a solid formulation (SF5′), which is the solid formulation (SF), (SF′), (SF″), (SF1), (SF2), (SF2′), (SF2″), (SF2″), (SF3), (SF3′), (SF3″), (SF4), (SF4′), (SF4″) or (SF4′″), wherein the content of the at least one hydrocolloid is 25 to 80 wt.-%, based on the total weight of the solid formulation.

Therefore, the present invention relates to a solid formulation (SF5″), which is the solid formulation (SF), (SF′), (SF″), (SF1), (SF2), (SF2′), (SF2″), (SF2″), (SF3), (SF3′), (SF3″), (SF4), (SF4′), (SF4″) or (SF4″), wherein the content of the at least one hydrocolloid is 25 to 75 wt.-%, based on the total weight of the solid formulation.

The solid formulation according to the present invention can comprise at least one oil. Suitable oils are such as coconut oil, sunflower oil, corn oil, MCT oil (medium chain triglycerides) or other vegetable oils.

When at least one oil is present, then the content of the at least one oil in the solid formulation is usually from 1 to 20 wt.-%, based on the total weight of the solid formulation, preferably from 2 to 15 wt.-%, more preferably from 3 to 10 wt.-%, based on the total weight of the solid formulation.

Therefore, the present invention relates to a solid formulation (SF6), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′) or (SF5″), wherein the at least one oil is chosen from the group consisting of coconut oil, sunflower oil, corn oil, MCT oil and other vegetable oils.

Therefore, the present invention relates to a solid formulation (SF7), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″) or (SF6), wherein the content of the at least one oil is 1 to 20 wt.-%, based on the total weight of the solid formulation.

Therefore, the present invention relates to a solid formulation (SF7′), which is the solid formulation (SF), (SF′), (SF″), (SF1), (SF2), (SF2′), (SF2″), (SF2″), (SF3), (SF3′), (SF3″), (SF4), (SF4′), (SF4″), (SF4′″), (SF5), (SF5′), (SF5″) or (SF6), wherein the content of the at least one oil is 2 to 15 wt.-%, based on the total weight of the solid formulation.

Therefore, the present invention relates to a solid formulation (SF7″), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″) or (SF6), wherein the content of the at least one oil is 3 to 10 wt.-%, based on the total weight of the solid formulation.

The solid formulation according to the present invention comprises tocopherol.

In the context of the present invention the term “tocopherol” encompasses DL-α-tocopherol ((all-rac)-α-tocopherol) and mixed tocopherol.

The content of the tocopherol in the solid formulation is from 0.5 to 10 wt.-%, based on the total weight of the solid formulation, preferably from 1 to 8 wt.-%, more preferably from 2 to 8 wt.-%, based on the total weight of the solid formulation.

Therefore, the present invention relates to a solid formulation (SF8), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′) or (SF7″), wherein the content of the tocopherol is 0.5 to 10 wt.-%, based on the total weight of the solid formulation.

Therefore, the present invention relates to a solid formulation (SF8′), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′) or (SF7″), wherein the content of the tocopherol is 1 to 8 wt.-%, based on the total weight of the solid formulation.

Therefore, the present invention relates to a solid formulation (SF8″), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′) or (SF7″), wherein the content of the tocopherol is 2 to 8 wt.-%, based on the total weight of the solid formulation.

The solid formulation according to the present invention comprises sodium ascorbate.

The content of the sodium ascorbate in the solid formulation is from 0.1 to 5 wt.-%, based on the total weight of the solid formulation, preferably from 0.5 to 4 wt.-%, more preferably from 1 to 4 wt.-%, based on the total weight of the solid formulation.

Therefore, the present invention relates to a solid formulation (SF9), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′) or (SF8″), wherein the content of the sodium ascorbate is 0.1 to 5 wt.-%, based on the total weight of the solid formulation.

Therefore, the present invention relates to a solid formulation (SF9′), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′) or (SF8″), wherein the content of the sodium ascorbate is 0.5 to 4 wt.-%, based on the total weight of the solid formulation.

Therefore, the present invention relates to a solid formulation (SF9″), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′) or (SF8″), wherein the content of the sodium ascorbate is 1 to 4 wt.-%, based on the total weight of the solid formulation.

Furthermore, the solid formulation according to the present invention can also comprise some residual water. The content is depending on the drying process, which has been used to dry the solid formulation.

The water content of the solid formulation according to the present invention is usually and preferably below 5 wt-%, based on the total weight of the solid formulation. More preferably the water content is below 4 wt-%, based on the total weight of the solid formulation.

Therefore, the present invention relates to a solid formulation (SF10), which is the solid formulation (SF), (SF′), (SF″), (SF1), (SF2), (SF2′), (SF2″), (SF2″), (SF3), (SF3′), (SF3″), (SF4), (SF4′), (SF4″), (SF4′″), (SF5), (SF5′), (SF5″), (SF6), (SF7), (SF7′), (SF7″), (SF8), (SF8′), (SF8″), (SF9), (SF9′) or (SF9″), wherein the water content of the solid formulation is below 5 wt-%, based on the total weight of the solid formulation.

Therefore, the present invention relates to a solid formulation (SF10′), which is the solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′) or (SF9″), wherein the water content of the solid formulation is below 4 wt-%, based on the total weight of the solid formulation.

The solid formulation according to the invention may further contain additional ingredients such powdering agent, anti-caking agents, e.g. tri-calcium phosphate and silicates, such as silicon dioxide or sodium aluminium silicate, flavours and colors.

The ingredients (i) to (v) as well as the residual water and the additional ingredients (such powdering agent, anti-caking agents, e.g. tri-calcium phosphate and silicates, such as silicon dioxide or sodium aluminium silicate, flavours and colors) are in the form of particles, which form the solid formulation according to the present invention.

Optionally, the particles, which for the solid formulation according to the present invention can be coated as well, which means they can have a coating layer.

The coating layer can be any commonly known coating layer. It could also be a powder coating layer. Such a powder coating can be made from starch (such as corn starch).

The coating layer can be up to 45 wt-% of the coated solid formulation, based on the total weight of the coated solid formulation. This means that at least 55 wt-% is the coated solid formulation is at least one solid formulation as defined above.

Therefore, the present invention relates to a coated solid formulation (CSF1), which is the solid formulation (SF), (SF′), (SF″), (SF1), (SF2), (SF2′), (SF2″), (SF2″), (SF3), (SF3′), (SF3″), (SF4), (SF4′), (SF4″), (SF4′″), (SF5), (SF5′), (SF5″), (SF6), (SF7), (SF7′), (SF7″), (SF8), (SF8′), (SF8″), (SF9), (SF9′), (SF9″), (SF10) or (SF10′) coated with a coating layer.

Therefore, the present invention relates to a solid formulation (CSF1′), which is the coated solid formulation (CSF1), wherein the coating layer is up to 45 wt-% of the coated solid formulation, based on the total weight of the coated solid formulation.

The solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (SF11) and (SF11′) as well as the coated solid formulation (CSF1) or (CSF1′) can be produced by commonly known method such as spray drying, powder catch process, freeze drying, granulation, agglomeration or extrusion.

The particle size of the solid formulation and coated solid formulation according to the present invention can vary according to the process, which is used to produce the (coated) solid formulation. A usual size is 10 μm up to 500 μm, preferably 10 μm up to 250 μm.

Therefore, the particle size of the (coated) solid formulation (SF), (SF′), (SF″), (SF1), (SF2), (SF2′), (SF2″), (SF2′″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and (CSF1′) is in the range of from 10 μm up to 500 μm, preferably from 10 μm up to 250 μm.

The (coated) solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and/or (CSF1′) is used to fortify staple food.

Preferably the (coated) solid formulation (SF), (SF′), (SF″), (SF1), (SF2), (SF2′), (SF2″), (SF2″), (SF3), (SF3′), (SF3″), (SF4), (SF4′), (SF4″), (SF4′″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and/or (CSF1′) is used to fortify flour, sugar, rice or condiments.

Preferably the (coated) solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and/or (CSF1′) is used to fortify flour.

Furthermore, the present invention relates to staple food fortified by the (coated) solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and/or (CSF1′).

Preferably the solid formulation is used to fortify flour, sugar, rice or condiments. More preferably the solid formulation is used to fortify flour.

Furthermore, the present invention relates to flour, sugar, rice or condiments fortified by the (coated) solid formulation (SF), (SF′), (SF″), (SF1), (SF2), (SF2′), (SF2″), (SF2″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and/or (CSF1′).

Furthermore, the present invention relates to flour fortified by the (coated) solid formulation (SF), (SF′), (SF″), (SF1), (SF2), (SF2′), (SF2″), (SF2′″), (SF3), (SF3′), (SF3″), (SF4), (SF4′), (SF4″), (SF4″), (SF5), (SF5′), (SF5″), (SF6), (SF7), (SF7′), (SF7″), (SF8), (SF8′), (SF8″), (SF9), (SF9′), (SF9″), (SF10), (SF10′), (CSF1) and/or (CSF1′).

The following examples illustrate the present invention.

All the parts and percentages in the Examples are related to the weight (when not otherwise stated) and the temperature is given in ° C. (when not otherwise stated).

### EXAMPLES

### Example 1

A matrix solution was prepared by dissolving 11.95 kg Hicap 100, 3.15 kg Gum Acacia and 0.89 kg sodium ascorbate in 23.0 kg water at 60° C. while stirring. 5.75 kg vitamin A palmitate (1.7 MioIU/g), 0.56 kg mixed tocopherols (70IP) were mixed and of this mixture 6.03 kg was added to the matrix solution. The mixture was emulsified until the particle size D (0,5) was below 1 μm as measured by Malvern Mastersizer 2000. The viscosity was adjusted by the addition of water and the emulsion was spray in corn starch. The powder was collected with an LOD of 5.1%. The final dry powder had the composition as shown in table 1 under Exp. 1.

### Example 2

A matrix solution was prepared by dissolving 267 g Hicap 100, and 10 g sodium ascorbate in 185 g water at 65° C. while stirring. 63.2 g vitamin A palmitate (1.7 MioIU/g), 20 g mixed tocopherols (70IP), and 40 g Coconut oil were mixed at 60° C. for 10 minutes and of this mixture 128 g was added to the matrix solution. The mixture was emulsified until the particle size D (0,5) was below 1 μm as measured by Malvern Mastersizer 2000. The viscosity was adjusted by the addition of water and the emulsion was spray dried to yield 214 g dry powder with an LOD of 2.3%. The final dry powder had the composition as shown in table 1 under Exp.2.

Examples 3 to 5 are comparison examples.

The dry powders (3 to 5) shown in table 1 were produced under the same conditions as example 2 by adapting the quantity of the ingredients in the matrix solution and the oil mixture.

TABLE 1
composition of the dry powder

| | Exp. 1 | Exp. 2 | Exp. 3 | Exp. 4 | Exp. 5 |
|---|---|---|---|---|---|
| Ingredients | [wt-%] | [wt-%] | [wt-%] | [wt-%] | [wt-%] |
| HiCap100 | 32.0 | 64.5 | 51.4 | 36.0 | 35.9 |
| Gum Acacia | 8.0 | 0.00 | 11.6 | 0.0 | 0.0 |
| Sodium Ascorbate | 2.5 | 2.40 | 1.4 | 2.5 | 0.5 |
| Maltodextrin DE19 IT | 0.0 | 0.0 | 9.5 | 37.2 | 31.3 |
| Corn starch | 35.5 | 0.0 | 0.0 | 0.0 | 0.0 |
| Vitamin A Palmitate | 15.5 | 15.8 | 15.9 | 15.9 | 15.9 |
| Coconut oil | 0.0 | 10.0 | 6.0 | 2.0 | 10.1 |
| Mixed tocopherol IP70 | 1.5 | 5.0 | 2.5 | 5.0 | 5.0 |
| residual water | 5.1 | 2.3 | 1.7 | 1.4 | 1.3 |

The produced dry powders were mixed in flour with a moisture content between 13.5 wt % and 14.5 wt % and stored in closed glass bottles for 21 days at 45° C.

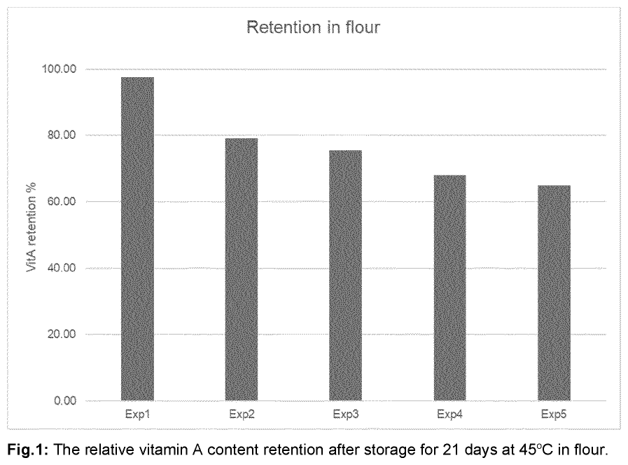

The vitamin A content was measured before and after storage and the relative retention was calculated and shown in FIG. 1.

It was surprisingly shown that the forms not comprising a starch hydrolysate had a similar or even improved stability compared to other powders containing various amount of starch hydrolysate.

1. A solid formulation comprising
(i) at least one fat soluble vitamin, and
(ii) at least one hydrocolloid, and
(iii) optionally at least one oil, and
(iv) tocopherol and
(v) sodium ascorbate,
with the proviso that the formulation does not comprise
(a) any BHT, and
(b) any starch hydrolysate, and
(c) any disaccharide.

2. Solid formulation according to claim 1, wherein the solid formulation is free of anydisaccharide chosen from the group consisting of sucrose, lactose, trehalose, cellobiose and chitobiose (preferably sucrose).

3. Solid formulation according to claim 1, wherein the solid formulation is free of any dextrin, any maltodextrin and any glucose syrup.

4. Solid formulation according to claim 1, wherein the at least one fat-soluble vitamin is chosen from the group consisting of vitamin A, vitamin D, Vitamin K and its derivatives thereof.

5. Solid formulation according to claim 1, wherein the at least one fat-soluble vitamin is chosen from the group consisting of vitamin A and derivatives thereof.

6. Solid formulation according to claim 1, wherein the content of the at least one fat-soluble vitamin is from 3 to 30 weight-% (wt.-%), based on the total weight of the solid formulation.

7. Solid formulation according to claim 1, wherein the at least one hydrocolloid is chosen from the group consisting of polysaccharides, gelatin of low bloom, medium bloom or high bloom from fish, pork or bovine, caseines/caseinates and other proteinaceous hydrocolloids.

8. Solid formulation according to claim 1, wherein the hydrocolloid is modified food starch.

9. Solid formulation according to claim 1, wherein the content of the at least one hydrocolloid is 25 to 85 wt.-%, based on the total weight of the solid formulation.

10. Solid formulation according to claim 1, wherein the at least one oil is chosen from the group consisting of coconut oil, sunflower oil, corn oil, MCT oil and other vegetable oils.

11. Solid formulation according to claim 1, wherein the content of the at least one oil is 1 to 20 wt.-%, based on the total weight of the solid formulation.

12. Solid formulation according to claim 1, wherein the content of the tocopherol is 0.5 to 10 wt.-%, based on the total weight of the solid formulation.

13. Solid formulation according to claim 1, wherein the content of the sodium ascorbate is 0.1 to 5 wt.-%, based on the total weight of the solid formulation.
